(19) United States
(12) Patent Application Publication
(10) Pub. No.: US 2008/0213345 A1
(43) Pub. Date: Sep. 4, 2008

(54) Delivering Iron to an Animal

(75) Inventors: Ning Hu, San Gabriel, CA (US); Gerard M. Jensen, Brea, CA (US); Craig Skenes, Lake Forest, CA (US); Stephanie Yang, Temple City, CA (US)

Correspondence Address: VIKSNINS HARRIS & PADYS PLLP, P.O BOX 111098, St. Paul, MN, 55111-1098, US

(73) Assignee: GILEAD SCIENCES, INC., Foster City, CA (US)

(21) Appl. No.: 11/662,048

(22) PCT Filed: Sep. 13, 2005
(86) PCT No.: PCT/US05/32674

§ 371 Date: Feb. 6, 2008

Related U.S. Application Data
(60) Provisional application No. 60/609,491, filed on Sep. 13, 2004.

(51) Int. Cl. A61K 9/10; A61K 33/26; A61P 7/06

(52) U.S. Cl. 424/450; 424/646; 424/647; 424/648

(57) ABSTRACT

The invention provides a method for delivery iron to an animal. This invention further provides a method for treating iron deficiency in an animal.

### CROSS-REFERENCE TO RELATED APPLICATION

This patent document claims priority to U.S. Application Ser. No. 60/609,491, filed on Sep. 13, 2004, which application is incorporated by reference herein.

### BACKGROUND

Liposomes are sub-micron spherical vesicles made of phospholipids and cholesterol that form a hydrophobic bilayer surrounding an aqueous core. These structures have been used with a wide variety of therapeutic agents and allow for a drug to be entrapped within the liposome based in part upon its own hydrophobic (bilayer entrapment) or hydrophilic properties (entrapment in the aqueous compartment).

Typically, encapsulating a drug in a liposome can alter the pattern of biodistribution and the pharmacokinetics for the drugs. In certain cases, liposomal encapsulation has been found to lower the toxicity of the drug. In particular, so-called long circulating liposomal formulations have been extensively studied. These liposomal formulations avoid uptake by the organs of the mononuclear phagocyte system, primarily in the liver and spleen. Such long-circulating liposomes may include a surface coat of flexible water soluble polymer chains that acts to prevent interaction between the liposome and plasma components that play a role in liposome uptake. Alternatively, such liposomes can be made without this coating, and instead with saturated, long-chain phospholipids and cholesterol.

Iron deficiency is the most common known form of nutritional deficiency. Its prevalence is highest among young children and women of childbearing age, particularly pregnant women. In children, iron deficiency causes developmental delays and behavioral disturbances, and in pregnant women, it increases the risk for a preterm delivery and delivering a low-birthweight baby. In the past three decades, increased iron intake among infants has resulted in a decline in childhood iron-deficiency anemia in the United States. As a consequence, the use of screening tests for anemia has become a less efficient means of detecting iron deficiency in some populations.

For women of childbearing age, iron deficiency has remained prevalent. In the human body, iron is present in all cells and has several vital functions. For example, it exists in the form of hemoglobin (Hb) as a carrier of oxygen to the tissues from the lungs; as myoglobin as a facilitator of oxygen use and storage in the muscles; as cytochromes as a transport medium for electrons within the cells; and as an integral part of enzyme reactions in various tissues. Too little iron can interfere with these vital functions and lead to morbidity and mortality.

Currently, the clinical management of iron deficiency involves treating patients with iron replacement products. Exemplary iron therapy options include oral iron, INFeD® (iron dextran injection), Venofer® (intravenous iron sucrose) and Ferrlecit® (intravenous sodium ferric gluconate complex in sucrose).

While oral iron supplementation is commonly used, it has several disadvantages that include side effects, poor compliance, poor absorption, and low efficacy in treating anemia due to the poor gastrointestinal absorption of iron. Gastrointestinal (GI) side effects include constipation, nausea, vomiting, and gastritis. Intravenous iron therapies have overcome the bioavailability issues associated with oral iron supplementation and have been shown to increase the efficacy of erythropoietin supplementation in stimulating red cell production. However, the use of INFeD® (iron dextran injection) has decreased in recent years because of the risk of anaphylactic reaction associated with the product, most likely due to the presence of dextran. Unlike INFeD® (iron dextran injection), Venofer® (intravenous iron sucrose) and Feirlecit® (intravenous sodium ferric gluconate complex in sucrose) pose little risk of inducing an anaphylactic reaction in patients, but there are adverse reactions associated with these products as well, including breathlessness, wheezing, abdominal or back pain, nausea, vomiting, and hypotension. These reactions are largely due to an overload of the transferrin molecule by administration of large doses of IV iron resulting in small amounts of ionized “free” iron remaining in the bloodstream. This can be exacerbated in persons with lower than normal levels of transferrin. In addition, excessive IV iron overload carries the risk of hemosiderosis, hepatic or cardiac organ dysfunction (from excess iron deposition), and bacterial infection.

Thus, there remains a need in the art for new iron therapy methods and products to help reduce the manifestations of iron deficiency (e.g., preterm births, low birthweight, and delays in infant and child development; or anemia in adults, e.g., from cancer or dialysis) and thus improve public health.

### SUMMARY OF CERTAIN EMBODIMENTS OF THE INVENTION

Certain embodiments of the invention provide a method to deliver iron to an animal, the method including administering to the animal a lipid-based dispersion including iron. Accordingly, certain embodiments of the invention provide a method to treat iron deficiency and associated diseases and conditions using a parenteral liposomal iron product that has the potential to avoid transferrin overload. Using the methods of the invention, iron is entrapped within a liposome during circulation, thus allowing for slow tissue uptake, e.g. to the liver and spleen. In addition, ionized “free” iron levels are low (essentially zero), with all iron typically either transferrin bound or entrapped in liposomes. This can provide for shorter infusion times or the ability to introduce more iron in the same time frame. The method is therefore especially amenable for use in patients that are hypotransferrinemic.

Accordingly, certain embodiments of the invention provide a method for treating iron deficiency in an animal including administering to the animal a lipid-based dispersion including iron, e.g., ferric ions such as ferric citrate, or ferrous ions. In one embodiment, the animal is a mammal, such as a human. In certain embodiments of the invention, the iron deficiency disease or condition is anemia.

### DETAILED DESCRIPTION

An “iron deficiency disease or condition” refers to a disease or a physiological condition associated with too little iron present in the body, either due to an inadequate diet, poor absorption of iron by the body, and/or loss of blood. Iron deficiency can also be related to lead poisoning in children, and can lead to iron deficiency anemia.

Quantitatively, for example, a total body iron average of less than approximately 3.8 g for a man or 2.3 g for a women, which is equivalent to 50 mg/kg body weight for a 75-kg man and 42 mg/kg body weight for a 55-kg woman, respectively, typically represents an iron deficient state. The total amount of iron in the body is determined by intake, loss, and storage of this mineral, using assays well-known to the art. For example, hemoglobin and serum ferritin assays are the common ways to test for anemia. In addition, serum transferrin receptor assays can be used to determine the presence of iron deficiency anemia.

Thus, “iron deficiency disease or condition” is meant to include, but is not limited to, a disease or condition characterized by low serum iron, increased serum iron-binding capacity, decreases serum ferritin, and/or decreased marrow iron stores, such as iron deficiency anemia, also referred to as hypoferric anemia, chronic anemia characterized by small, pale red blood cells and iron depletion, anemia of chronic blood loss, hypochromic-microcytic anemia, chlorosis, hypochromic anemia of pregnancy, infancy, and childhood, posthemorrhagic anemia, and anemia associated with cancer or dialysis.

“Anemia” refers to any condition in which the number of red blood per cubic mm, the amount of hemoglobin in 100 ml of blood, or the volume of packed red blood cells per 100 ml of blood are less than normal. Anemia can be classified, for example, into types such as blood loss anemias, anemias associated with problems of cell and pigment production, megaloblastic anemias, corpuscular hemolytic anemias, anemias associated with increased hemolysis, serogenic hemolytic anemias, and toxic hemolytic anemias.

In certain embodiments, the animal is a mammal, e.g., a human. In certain embodiments, the animal is at high risk for iron deficiency. In certain embodiments, the mammal is a female, e.g., a female of childbearing age such as a pregnant female. In certain embodiments, the female is a lactating female. In certain embodiments, the animal is a child. In certain embodiments, the child is less than about 18 years old. In certain embodiments, the child is about 1 week, about 1 month, about 6 months, or about 1, 2, 3, 4, 5, 6, 7, 8, 9, 10, 11, 12, 13, 14, 15, 16, 17, or 18 years old. In certain embodiments, the child is about 0-6 months old. In certain embodiments, the child is about 6-9 months old. In certain embodiments, the child is about 6-12 months old. In certain embodiments, the child is about 1-4 years old. In certain embodiments, the child is an adolescent child. In certain embodiments, the animal is an overweight animal.

The lipid-based dispersions of the present invention include a lipid layer including liposome forming lipids. Typically, the lipid includes at least one phosphatidyl choline which provides the primary packing/entrapment/structural element of the liposome. Typically, the phosphatidyl choline includes mainly C₁₆ or longer fatty-acid chains. Chain length provides for both liposomal structure, integrity, and stability. Optionally, in one embodiment, the fatty-acid chains can have at least one double bond.

As used herein, the term “phosphatidyl choline” includes Soy PC, Egg PC dielaidoyl phosphatidyl choline (DEPC), dioleoyl phosphatidyl choline (DOPC), distearoyl phosphatidyl choline (DSPC), hydrogenated soybean phosphatidyl choline (HSPC), dipalmitoyl phosphatidyl choline (DPPC), 1-palmitoyl-2-oleo phosphatidyl choline (POPC), dibehenoyl phosphatidyl choline (DBPC), and dimyristoyl phosphatidyl choline (DMPC), and mixtures thereof.

As used herein, the term “Soy-PC” refers to phosphatidyl choline compositions including a variety of mono-, di-, tri-unsaturated, and saturated fatty acids. Typically, Soy-PC includes palmitic acid present in an amount of about 12% to about 33% by weight; stearic acid present in an amount of about 3% to about 8% by weight; oleic acid present in an amount of about 4% to about 22% by weight; linoleic acid present in an amount of about 60% to about 66% by weight; and linolenic acid present in an amount of about 5% to about 8% by weight.

As used herein, the term “Egg-PC” refers to a phosphatidyl choline composition including, but not limited to, a variety of saturated and unsaturated fatty acids. Typically, Egg-PC includes palmitic acid present in an amount of about 34% by weight; stearic acid present in an amount of about 10% by weight; oleic acid present in an amount of about 31% by weight; and linoleic acid present in an amount of about 18% by weight.

As used herein, the terms “DEPC” and “DOPC” refer to phosphatidyl choline compositions including C₁₈ fatty acids with one unsaturation and wherein the fatty acid is present in an amount from about 90% to about 100%, preferably, about 100%.

Cholesterol typically provides stability to the liposome. The ratio of phosphatidyl choline to cholesterol is typically from about 0.5:1 to about 4:1 by mole ratio. Preferably, the ratio of phosphatidyl choline to cholesterol is from about 1:1 to about 2:1 by mole ratio. More preferably, the ratio of phosphatidyl choline to cholesterol is about 2:1 by mole ratio.

As used herein the term “total lipid” includes phosphatidyl cholines and any anionic phospholipid present.

The liposome may also include physiologically acceptable salts to maintain isotonicity with animal serum. Any pharmaceutically acceptable salt that achieves isotonicity with animal serum is acceptable, such as NaCl.

### Anionic Phospholipid

An anionic phospholipid may be used and typically provides a Coulombic character to the liposomes. This can help stabilize the system upon storage and can prevent fusion or aggregation or flocculation; it can also facilitate or enable freeze drying. It can also help direct recticuloendothelial system targeting. Phospholipids in the phosphatidic acid, phosphatidylglycerol, and phosphatidylserine classes (PA, PG, and PS) are particularly useful in the formulations of the invention. The anionic phospholipids typically include mainly C₁₆ or larger fatty-acid chains.

In one embodiment, the anionic phospholipid is selected from Egg-PG (Egg-Phosphatidyglycerol), Soy-PG (Soy-Phosphatidylglycerol), DSPG (Distearoyl Phosphatidyglycerol), DPPG (Dipalmitoyl Phosphatidyglycerol), DEPG (Dielaidoyl Phosphatidyglycerol), DOPG (Dioleoyl Phosphatidyglycerol), DSPA (Distearoyl Phosphatidic Acid), DPPA (Dipalmitoyl Phosphatidic Acid), DEPA (Dielaidoy Phosphatidic Acid), DOPA (Dioleoyl Phosphatidic Acid), DSPS (Distearoyl Phosphatidylserine), DPPS (Dipalmitoyl Phosphatidylserine), DEPS (Dielaidoy Phosphatidylserine), and DOPS (Dioleoyl Phosphatidylserine), and mixtures thereof. In another embodiment the anionic phospholipid is DSPG.

### Preparation of Liposomes

The liposomes of the invention include a lipid layer of phospholipids and cholesterol. Typically, the ratio of phospholipid to cholesterol is sufficient to form a liposome that will not substantially rapidly dissolve or disintegrate once administered to the patient. The phospholipids and cholesterol are dissolved in suitable solvent or solvent mixtures. After a suitable amount of time, the solvent is removed via vacuum drying and/or spray drying. The resulting solid material can be stored or used immediately.

Subsequently, the resulting solid material is hydrated in aqueous solution containing an appropriate concentration of iron at an appropriate temperature, resulting in multilameller vesicles (MLV). The solutions containing MLV can be size-reduced via homogenization to form Small Unilameller Vesicles (SUVs) with the drug passively entrapped within the formed SUVs. The resulting liposome solution can be purified of unencapsulated iron, for example by chromatography or filtration, and then filtered for use.

### Iron

As used herein, the term “iron” includes any pharmaceutically acceptable iron compound that can be used in the methods of the present invention, including an iron supplement, e.g., iron II (ferrous) or iron III (ferric) supplements, such as ferrous sulfate, ferric chloride, ferrous gluconate, ferrous lactate, ferrous tartrate, iron-sugar-carboxylate complexes, ferrous fumarate, ferrous succinate, ferrous glutamate, ferric citrate, ferrous citrate, ferrous pyrophosphate, ferrous cholinisocitrate, and ferrous carbonate, and the like. In one embodiment the iron is ferric citrate.

### Relative Amounts

The present invention also provides liposomes, dispersions, compositions and formulations as described herein useful, for example, for delivering iron to an animal.

In one embodiment, the lipid-based dispersion includes from 0.05 to 60% anionic phospholipid by molar ratio relative to phosphatidyl choline.

In one embodiment, the weight ratio of total lipid (phosphatidyl choline+anionic phospholipid) to iron is greater than 1:1.

In another embodiment, the weight ratio of total lipid (phosphatidyl choline+anionic phospholipid) to iron is greater than 5:1.

In another embodiment, the weight ratio of total lipid (phosphatidyl choline+anionic phospholipid) to iron is greater than 10:1.

In another embodiment, the weight ratio of total lipid (phosphatidyl choline+anionic phospholipid) to iron is greater than 20:1.

In one embodiment, the invention provides a formulation including iron in a liposome that includes HSPC:Cholesterol:DSPG in a ratio of about 2:1:0.2.

In another embodiment, the invention provides a formulation including iron in a liposome that includes HSPC:Cholesterol:DSPG in a ratio of about 2:1:0.3.

In another embodiment, the invention provides a formulation including iron in a liposome that includes HSPC:Cholesterol:DSPG in a ratio of about 2:1:0.4.

In another embodiment, the invention provides a formulation including iron in a liposome that includes DEPC:Cholesterol in a ratio of about 2:1.

In another embodiment, the invention provides a formulation including iron in a liposome that includes DEPC:Cholesterol:DSPG in a ratio of about 2:1:0.1.

In another embodiment, the invention provides a formulation including iron in a liposome that includes DOPC:Cholesterol in a ratio of about 2:1.

In one embodiment of the invention, the lipid-based dispersion can have one or more phosphatidyl choline, cholesterol, iron and, optionally, one or more anionic phospholipids. For example, in one embodiment, the lipid-based dispersion can have a mole ratio of phosphatidyl choline to cholesterol from about 0.5 to 1, to about 4:1, e.g., a mole ratio of phosphatidyl choline to cholesterol from about 1 to 1, to about 2:1. The phosphatidyl choline can be, for example, DEPC, DOPC, DSPC, HSPC, DMPC, DPPC or mixtures thereof. For example, the phosphatidyl choline can be HSPC, DOPC, DEPC and mixtures thereof. For example, in certain embodiments of the invention, the lipid-based dispersion can have HSPC:Cholesterol:DSPG in a ratio of about 2:1:0.3; HSPC:Cholesterol:DSPG in a ratio of about 2:1:0.2; DEPC:Cholesterol:DSPG in a ratio of about 2:1:0.1; or DOPC:Cholesterol in a ratio of 2:1.

### Formulations

The formulations of the invention can be administered to an animal host, e.g., a mammalian host, such as a human patient in a variety of forms adapted to the chosen route of administration. For example, they can be formulated to be administered parenterally. Moreover, the lipid-based dispersions can be formulated for subcutaneous, intramuscular, intravenous, or intraperitoneal administration by infusion or injection. These preparations may also contain a preservative to prevent the growth of microorganisms, buffers, or anti-oxidants in suitable amounts.

Useful dosages of the formulations of the invention can be determined by comparing their in vitro activity, and in vivo activity in animal models. Methods for the extrapolation of effective dosages in mice, and other animals, to humans are known to the art; for example, see U.S. Pat. No. 4,938,949.

The lipid-based dispersions of the present invention typically have about 1 mg/mL to about 10 mg/mL iron. Generally, the concentration of iron in a unit dosage form of the invention will typically be from about 0.5-50% by weight of the composition, preferably from about 2-20% by weight of the composition.

The amount of iron required for use in treatment will vary not only with particular type of iron compound or supplement, but also with the route of administration, the nature of the condition being treated and the age and condition of the patient; the amount required will be ultimately at the discretion of the attendant physician or clinician.

The desired amount of a formulation may conveniently be presented in a single dose or as divided doses administered at appropriate intervals, for example, as two, three, four or more sub-doses per day. The sub-dose itself may be further divided, e.g., into a number of discrete loosely spaced administrations.

Pharmacokinetic data (plasma concentration vs. time post injection) for iron in a formulation of the invention and for the free iron can be determined in an array of known animal models. For example, it can be determined in rats using Test A.

### Test Method A—Pharmacokinetics (PK)

Pharmacokinetic data (plasma concentration vs. time post injection) is obtained for one dose per liposome formulation and the corresponding free drug. Sprague Dawley or Wistar rats, female, are used, weighing about 150 g. Typically there are 6 rats per dose group. Plasma pulls of 200 microliters (sampling from the orbital sinus) are collected in EDTA tubes, with samples frozen prior to chemical analysis of the drug.

One ml of blood with a hemoglobin concentration of 12 g/dL would contain 120 mg hemoglobin/ml. Hemoglobin is 0.34% iron. Therefore, blood with 120 mg hb/ml would contain 0.41 mg Fe/ml in hemoglobin. A loss of 200 ml blood with 12 g hemoglobin/dL would result in a loss of 82 mg Fe.

The maximum tolerated dose for iron in a formulation of the invention and for free iron can be determined in an array of known animal models. For example, it can be determined using Test B.

### Test Method B—Maximum Tolerated Dose (TD)

Nude mice (NCr.nu/nu-mice) are administered each liposomal formulation, and free iron, by I.V. administration and the maximum tolerated dose (MTD) for each formulation is then determined. Typically, a range of doses are given until an MTD was found, with 2 mice per dose group. Estimate of MTD is determined by evaluation of body weight, lethality, behavior changes, and/or signs at autopsy. Typical duration of the experiment is observation of the mice for four weeks, with body weight measurements twice per week.

Alternatively, the MTD of bolus I.V. or I.P. doses can be evaluated in a hypotransferrinemic mouse (e.g., the heterozygous Trfr⁻ mouse described in Trenor et al. (2000)). Alternatively, a maximum infusion rate can be determined for each formulation in an animal by I.V. infusion.

The invention is further defined by reference to the following examples describing the preparation of formulations of the invention. It will be apparent to those skilled in the art, that many modifications, both to materials and methods, may be practiced without departing from the purpose and interest of this invention.

### EXAMPLES

### General Procedure of Liposome Preparation

Lipid films or lipid spray dried powder containing various phospholipids including hydrogenated soy phosphatidyl choline (HSPC), dioleoyl phosphatidyl choline (DOPC), dielaidoyl phosphatidyl choline (DEPC), cholesterol (Chol) and distearoylphosphatidylglycerol (DSPG) at the following mole ratios were prepared.

HSPC:Chol:DSPG at a) 2:1:0.2 b) 2:1:0.3 c) 2:1:0.4

DOPC:Chol at a) 2:1

DEPC:Chol:DSPG at a) 2:1:0.1

### Lipid Film Preparation

A stock solution of each lipid component was made in a chloroform:methanol 1:1 (v/v) organic solvent system. The final concentration of each lipid component was 50 mg/ml. Lipid solutions were pipetted according to the designed mole ratio and were mixed in a conical tube. The solvent was then removed by running nitrogen through the solution while the solution was heated in heat block with temperature set at 65° C. The formed lipid film was then left in a desicator under vacuum to remove residual organic solvent until used, and for not less than 48 hours.

### Spray Dried Lipid Powder Preparation

All the lipid components were weighed out and were mixed in a round bottom flask. A chloroform:methanol 1:1 (v/v) solvent was added to the lipid powder with a final lipid concentration of around 100 mg/ml. The lipid solution was then spray dried to form lipid powder using a YAMATO GB-21 spray drier at a designed parameter setting. The residual solvent in the lipid powder was removed by drying under vacuum for three to five days.

### Ferric Citrate Stock Solution Preparation

A ferric citrate stock solution at concentration of 600 mg/mL was prepared by dissolving ferric citrate powder in water for injection at room temperature.

Preparation of Liposomes by Probe Sonication from Either Lipid Film or Spray Dried Lipid Powder

Lipid film or lipid powder was weighed out and hydrated with a 600 mg/mL ferric citrate stock solution in a 65° C. water bath at lipid concentrations of approximately 200 mg/ml. The hydrated solution was subjected to probe sonication until the solution became translucent. A typical temperature of sonication was 65° C. and a typical sonication time was 15 to 20 minutes. After completion of sonication, i.e. formation of liposomes, the solution was diluted 50-fold with 9% sucrose solution or with 9% sucrose solution containing 1 mM-10 mM NH₄Cl with pH adjusted to 5.0-7.5. The unencapsulated free iron in the resulting liposome solution was removed by ultrafiltration/buffer exchange with 9% sucrose solution or with 9% sucrose solution containing 1 mM-10 mM NH₄Cl with pH adjusted to 5.0-7.5. Following buffer exchange, the solution was concentrated back 50 fold. The resulting solution was sterile filtered using a 0.2 um PES (polyether sulfone) filter and aseptically stored at 2-8° C.

Preparation of Liposomes by Homogenization from Spray Dried Lipid Powder

Lipid powder was weighed out and hydrated with a 600 mg/mL ferric citrate stock solution in a 65° C. water bath at lipid concentration approximately 200 mg/ml. The hydrated solution was subjected to homogenization using a Niro homogenizer at 10,000 PSI at 65° C. until the solution became translucent. Typically, the solution was pumped through the homogenizer continuously for about 25-30 passes, or until the solution became translucent. After completion of homogenization, i.e. liposome formation, the liposomal solution was diluted 50-fold with 9% sucrose solution or with 9% sucrose solution containing 1 mM-10 mM NH₄Cl with pH adjusted to 5.0-7.5. The unencapsulated free iron in the resulting liposome solution was removed by ultrafiltration/buffer exchange with 9% sucrose solution or with 9% sucrose solution containing 1 mM-10 mM NH₄Cl with pH adjusted to 5.0-7.5. Following buffer exchange, the solution was concentrated back 50 fold. The resulting solution was sterile filtered using a 0.2 um PES (polyether sulfone) filter and aseptically stored at 2-8° C.

### Example 1

Liposomes were prepared as described above. Characterization data for representative liposomes is shown in Table 1.

TABLE 1

| | | Mole | | Liposome | | Size | | |
|---|---|---|---|---|---|---|---|---|
| No. | Lipid Formulation | Ratio | Ultrafiltration buffer | Formation | A600 | (nm) | Volume % | pH |
| 1 | HSPC/Chol/DSPG | 2:1:.02 | 9% sucrose | Sonication | 1.2 | 68 | 100 | 5.2 |
| 2 | HSPC/Chol/DSPG | 2:1:0.3 | 9% sucrose | Sonication | 1.1 | 68 | 100 | 5.1 |
| 3 | HSPC/Chol/DSPG | 2:1:0.4 | 9% sucrose | Sonication | 1.4 | 91 | 100 | 5.0 |
| 4 | HSPC/Chol/DSPG | 2:1:0.3 | 9% sucrose | homogenization | 1.0 | 67 | 100 | 5.5 |
| 5 | HSPC/Chol/DSPG | 2:1:0.3 | 9% sucrose/1 mM NH₄Cl | Homogenization | 1.2 | 56 | 100 | 6.4 |
| 6 | HSPC/Chol/DSPG | 2:1:0.3 | 9% sucrose/5 mM NH₄Cl | Homogenization | 1.2 | 57 | 100 | 6.3 |
| 7 | HSPC/Chol/DSPG | 2:1:0.3 | 9% sucrose/10 mM NH₄Cl | Homogenization | 1.3 | 53 | 100 | 6.4 |
| 8 | HSPC/Chol/DSPG | 2:1:0.3 | 9% sucrose | Homogenization | 1.3 | 65 | 100 | 5.5 |
| 9 | HSPC/Chol/DSPG | 2:1:0.3 | 9% sucrose/1 mM NH₄Cl pH 5.0 | Homogenization | 1.4 | 59 | 100 | 5.3 |
| 10 | HSPC/Chol/DSPG | 2:1:0.3 | 9% sucrose/1 mM NH₄Cl pH 6.5 | Homogenization | 1.5 | 59 | 100 | 6.4 |
| 11 | HSPC/Chol/DSPG | 2:1:0.3 | 9% sucrose/1 mM NH₄Cl pH 7.5 | Homogenization | 1.4 | 61 | 100 | 7.1 |
| 12 | DOPC/Chol | 2:1 | 9% sucrose | Sonication | 0.7 | 77 | 100 | 5.4 |
| 13 | DEPC/Chol | 2:1 | 9% sucrose/10 mM NH₄Cl pH 6.5 | Sonication | 0.7 | 72 | 100 | 6.4 |
| 14 | HSPC/Chol/DSPG | 4:1:0.1 | 9% sucrose/1 mM NH₄Cl pH 6.5 | Homogenization | 0.8 | 38 | 100 | 6.4 |
| 15 | DEPC/Chol/DSPG | 2:1:0.1 | 9% sucrose/1 mM NH₄Cl pH 6.5 | Homogenization | 1.1 | 44 | 100 | 6.0 |

### Example 2

The following illustrate representative pharmaceutical dosage forms, containing a lipid-based dispersion of the invention, for therapeutic or prophylactic use in animals (e.g. humans).

TABLE 2

| | mg/ml |
|---|---|
| (i) Injection 1 (1 mg/ml) | |
| ‘Iron’ | 1.0 |
| Phosphatidyl choline | 40 |
| Cholesterol | 10 |
| Sucrose | 90 |
| 0.1 N Sodium hydroxide solution | q.s. |
| (pH adjustment to 7.0-7.5) | |
| Water for injection | q.s. ad 1 mL |
| (ii) Injection 2 (10 mg/ml) | |
| ‘Iron’ | 10 |
| Phosphatidyl choline | 60 |
| Cholesterol | 15 |
| Anionic Phospholipid | 3 |
| 0.1 N Sodium hydroxide solution | q.s. |
| (pH adjustment to 7.0-7.5) | |
| sucrose | 90 |
| Water for injection | q.s. ad 1 mL |

The above formulations may be obtained by conventional procedures well known in the pharmaceutical art. All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method of delivering iron to an animal, comprising administering to the animal a lipid-based dispersion comprising iron.

2. The method of claim 1, wherein the lipid-based dispersion comprises ferric ions.

3. The method of claim 1, wherein the lipid-based dispersion comprises ferric citrate.

4. The method of claim 1, wherein the lipid-based dispersion comprises ferrous ions.

5. A method of delivery iron to an animal comprising administering to the animal a lipid-based dispersion comprising a) one or more phosphatidyl choline; b) cholesterol; c) iron, and optionally d) one or more anionic phospholipids.

6. The method of claim 5, wherein the lipid-based dispersion has a mole ratio of phosphatidyl choline to cholesterol from about 0.5:1 to about 4:1.

7. The method of claim 6, wherein the lipid-based dispersion has mole ratio of phosphatidyl choline to cholesterol from about 1:1 to about 2:1.

8. The method of claim 5, wherein the lipid-based dispersion has a mole ratio of phosphatidyl choline to cholesterol that is about 2:1.

9. The method of claim 5 wherein the phosphatidyl choline is selected from DEPC, DOPC, DSPC, HSPC, DMPC, and DPPC, and mixtures thereof.

10. The method of claim 9, wherein the phosphatidyl choline is selected from HSPC, DOPC, and DEPC, and mixtures thereof.

11. The method of claim 9, wherein the lipid-based dispersion comprises HSPC:Cholesterol:DSPG in a ratio of about 2:1:0.4.

12. The method of claim 9, wherein the lipid-based dispersion comprises HSPC:Cholesterol:DSPG in a ratio of about 2:1:0.3.

13. The method of claim 9, wherein the lipid-based dispersion comprises HSPC:Cholesterol:DSPG in a ratio of about 2:1:0.2.

14. The method of claim 9, wherein the lipid-based dispersion comprises DEPC:Cholesterol:DSPG in a ratio of about 2:1:0.1.

15. The method of claim 9, wherein the lipid-based dispersion comprises DOPC:Cholesterol in a ratio of about 2:1.

16. The method of claim 5, wherein the lipid-based dispersion comprises small unilameller vesicles (SUVs).

17. The method of claim 5, wherein the lipid-based dispersion comprises multilameller vesicles (MLVs).

18. The method of claim 5, wherein the lipid-based dispersion comprises SUVs and MLVs.

19. The method of claim 5, wherein the animal has an iron deficiency disease or condition.

20. The method of claim 19, wherein the iron deficiency disease or condition comprises anemia.

21. The method of claim 5, wherein the animal is a mammal.

22. The method of claim 21, wherein the mammal is a human.

23. The method of claim 22, wherein the human is a female.

24. The method of claim 21, wherein the mammal is a pregnant mammal.

25. The method of claim 21, wherein the mammal is a lactating mammal.

26. The method of claim 22, wherein the human is less than about 18 years old.

27. The method of claim 22, wherein the human is about 0-6 months old.

28. The method of claim 22, wherein the human is about 6-9 months old.

29. The method of claim 22, wherein the human is about 6-12 months old.

30. The method of claim 22, wherein the human is about 1-4 years old.

31. The method of claim 22, wherein the lipid-based dispersion is administered parenterally.

32-46. (canceled)

47. The method of claim 1, wherein the lipid-based dispersion comprises one or more phosphatidyl choline.
